(19) United States
(12) Patent Application Publication
(10) Pub. No.: US 2005/0119661 A1
(43) Pub. Date: Jun. 2, 2005

(54) Methods and systems for intraoperative measurement of soft tissue constraints in computer aided total joint replacement surgery

(76) Inventors: Antony J. Hodgson, Vancouver (GB); Scott J. Illsley, Hollywood, FL (US)

Correspondence Address: DORSEY & WHITNEY LLP;INTELLECTUAL PROPERTY DEPARTMENT, SUITE 3400, 1420 FIFTH AVENUE, SEATTLE, WA, 98101, US

(21) Appl. No.: 10/495,850

(22) PCT Filed: Nov. 14, 2002
(86) PCT No.: PCT/US02/36719

(30) Foreign Application Priority Data
Nov. 14, 2001 (US) 60331307

(51) Int. Cl. A61B 17/88; A61F 2/46

(52) U.S. Cl. 606/90; 606/102; 623/908

(57) ABSTRACT

Methods and systems are described to quantitatively determine the degree of soft tissue constraints on knee ligaments and for properly determining placement parameters for prosthetic components in knee replacement surgery that will minimize strain on the ligaments. In one aspect, a passive kinetic manipulation technique is used in conjunction with a computer aided surgery (CAS) system to accurately and precisely determine the length and attachment sites of ligaments. These manipulations are performed after an initial tibial cut and prior to any other cuts or to placement of any prosthetic component. In a second aspect, a mathematical model of knee kinematics is used with the CAS system to determine optimal placement parameters for the femoral and tibial components of the prosthetic device that minimizes strain on the ligaments.

### CROSS-REFERENCE TO RELATED APPLICATION

This application claims priority to U.S. provisional patent application No. 60/331,307, filed Nov. 14, 2001.

### TECHNICAL FIELD

The invention relates to methods and systems for determining ligament attachment sites and lengths for proper soft-tissue balancing when orienting prosthetic components in joint replacement surgery, particularly in total knee replacement surgery; to methods for determining prosthetic component placement parameters; and to computer aided systems configured with instructions for facilitating the same.

### BACKGROUND OF THE INVENTION

In joint replacement surgery, exemplified by total knee replacement surgery, the surgeon attempts to restore limb alignment by removing the damaged surfaces of the joint and replacing them with metal and plastic (or sometimes ceramic) components. These components must be precisely aligned to maximize the implant's lifespan. The components are held together by soft tissue structures surrounding the joint. In the knee, the primary soft tissue structures are the posterior cruciate, the lateral collateral and the medial collateral ligaments. These ligaments must be properly balanced to match the bone cuts—they cannot be too long or the knee will separate (a problem known as instability) and they cannot be too short or they may rupture when strained.

Currently, soft tissue balancing is considered an imprecise art because there are few ways to quantify the appropriateness of the soft tissue balancing that a surgeon does. Furthermore, the few existing techniques for quantifying balance are applied after the bone cuts are complete, so the state of the soft tissue cannot enter into prior planning of the surgical process. Because problems with soft tissue balancing represent one of the major unsolved problems in knee surgery, there is considerable interest in developing tools to assist with this process.

Conventional prior art methods for orienting prosthetic components involve measuring a deviation from rectangularity of the space created by the distal femoral and tibial plateau resections when the limb is placed in extension or by the posterior femoral and tibial plateau resections when the limb is placed in flexion. Some of these prior art methods are described in articles by Andriacchi, T. P., Stanwyck, T. S., and Galante, J. O., entitled Knee biomechanics and total knee replacement, J Arthroplasty, 1(3) 211-9, 1986; Attfield, S. F., Warren˜Forward, M., Wilton, T., and Sambatakakis, A., entitled Measurement of soft-tissue imbalance in total knee arthroplasty using electronic instrumentation, Medical Engineering and Physics, 16(6) 501-505, 1994; and CAOS, Abstracts from CAOS-International 2001 First Annual Meeting of the International Society for Computer Assisted Orthopaedic Surgery Davos, Switzerland, Feb. 7-20, 2001, Comput Aided Surg, 6(2) 111-30, 2001. Other methods use pressure films or sensors after a trial component part is placed to detect evidence of overly tight ligaments as described in an article by Chao, E. Y.; Neluheni, E. V.; Hsu, R. W.; and Paley, D., entitled Biomechanics of Malalignment, 25(3) 379-386, 1994; and an article by Chen, E.; Ellis, R. E.; and Bryant, J. T., entitled A Strain-Energy Model of Passive Knee Kinematics for the Study of Surgical Implantation Strategies, MICCAI 2000, 1086-1095, 2000. Another method described by Martelli, S.; Ellis, R. E.; Marcacci, M.; and Zaffagnigi, S., in an article entitled Total knee arthroplasty kinematics, Computer simulation and intraoperative evaluation, J Arthroplasty, 13(2) 145-55, 1998, predicts the extent of overtensioning of the ligaments based on intraoperative digitization of the ligament origins and insertions. While this could in principle be applied at the planning stage of the procedure, methods and systems for doing so have not been described to date. An article by Sarin V K and Stulberg D, entitled Abstracts from LAOS-international 2001 First Annual Meeting of the International Society for Computer Assisted Orthopaedic Surgery Davos, Switzerland, Feb. 7-10, 2001, Comput. Aided Surg., 6(2) 111-130, 2001, describes a position measurement system integrated into a computer-assisted surgical system (CAS) to measure the extent of varus or valgus looseness after the components had been placed. Such post operative methods do not aid in the initial planning of the surgical procedure. Moreover, it is currently unclear how accurately a digitized center for the ligament origins or insertions represent actual constraints because ligaments consist of a large number of fibers. The load borne by the fibers may well shift throughout the range of motion of the knee and the fibers themselves typically wrap around bony portions of the knee, so the anatomical centers of the ligament origins and insertions may not be particularly good approximations of the effective positions of the constraints they provide.

One of the major goals of total knee arthroplasty (TKA) is the restoration of normal knee kinematics. This is dependent on the geometry of the prosthetic components, the placement of the components and the ligament balance as described in the above-cited articles by Andriacchi, et al; Chao, et al; and in an article by Faris, P. M., entitled Soft tissue balancing and total knee arthroplasty, in Knee Surgery, pp. 1385-1389. For passive knee kinematics as observed by a surgeon in the operating room, the components are kept in contact by the tensile forces exerted by the surrounding ligaments. Thus, for a given component placement position and geometry the passive kinematics are governed by the interaction of the contacting surfaces of the femoral and tibial components under the influence of the surrounding ligaments. Obtaining appropriate ligament balance during the procedure is a requirement for a successful implantation. At the present time, however, objective methods are not commonly used to quantify the techniques for balancing the surrounding ligaments during knee surgery. This makes it difficult to investigate the effect specific ligament alterations have on outcomes and to compare the techniques of different surgeons.

Attempts have been made by many researchers to quantify the degree of ligamenteous balance intraoperatively. Some of these attempts are described in previously cited articles by Attfield, et al; CAOS; Sambatakakis, A.; and Attfield, S. F.; and in articles by Newton, G., entitled Quantification of soft-tissue imbalance in condylar knee arthroplasty, Journal of Biomechanical Engineering, 15(4) 339-343, 1992; Takahashi, T.; Wada, Y.; and Yamamoto, H., entitled Soft tissue balancing with pressure distribution during total knee arthroplasty, J Bone and Joint Surgery, 79B(2) 235-239, 1997; and Wallace, A. L.; Harris, M. L.; Walsh, W. R; and Bruce, W. J. M., entitled Intraoperative Assessment of Tibiofemoral Contact Stresses in Total Knee Arthroplasty, J Arthroplasty, 13(8) 923-927, 1998. These methods are generally based on static measures of contact pressure and/or relative tension in the ligaments and focus mainly on imbalance in the frontal plane (varus/valgus). They generally measure the imbalance apparent in the flexion and extension gaps formed after both femoral and tibial bone cuts have been completed. The knee is “balanced” when equally spaced, rectangular gaps have been obtained as depicted in FIGS. 1 and 2. Such methods do not assess the overall kinematics of the knee throughout the range of motion, nor do they generally check the crucial midflexion gap illustrated in FIG. 3.

Computer models can be beneficial in exploring the knee kinematics throughout the range of motion. The previously mentioned Martelli et al article describes a strain energy model to analyze the passive kinematics as an instantaneous quasi-static solution to ligament strain energy minimization, similar to the work by Essinger, J. R.; Leyvraz, P. F.; Heegard, J. H.; and Robertson, D. D., described in an article entitled A mathematical model for the evaluation of the behavior during flexion of condylar-type knee prostheses, J Biomech, 22(11-12) 1229-41, 1989. For this model, the inputs included the knee joint geometry, the state of individual ligaments, the surgeon's choice of implant placement and the degree of flexion of the knee. The outputs of the model were the location of the contact point between the components' bearing surfaces and the resultant state of the ligaments. This provided the kinematics of the knee joint and the resulting strain in each ligament throughout the specified range of motion. This model was later extended from 2D to 3D, as described in the previously cited article by Chen et al, who also modeled each ligament as a set of finite ligament fibers to simulate their varying activity at different flexion angles.

Accuracy of the strain energy model is sensitive to (i.e., dependent on) the accuracy of data input regarding the locations of ligament attachment sites (origin and insertion sites) and the lengths of the ligaments. The geometries of prosthetic components are well known and their placement may be accurately specified, however, obtaining accurate information regarding the ligament lengths and attachment sites is difficult due to the limitations introduced by the intraoperative environment. During surgery, access to the ligament origin and insertion sites is limited and overlying soft tissue and bodily fluids hamper clear visualization. The attachment sites of the ligaments cover a finite area of bone making it difficult to identify a specific functional site of attachment. The Martelli et al article describes a technique for performing measurements using engineering calipers and claims this to be the most critical step in building an individual model of the knee. They reported standard deviations for lengths of the PCL, MCL and LCL across a set of subjects to be 3.27 mm, 6.86 mm and 4.62 mm respectively, using the caliper measurement method.

Computer models lend themselves to implementation using the hardware required for modern day CAS. CAS systems have recently been made commercially available for knee arthroplasty and show improvements in registration accuracy, which are described in the Sambatakakis et al. article. The first generation of computer assisted total knee surgery systems has mainly concentrated on registration of bone cuts to obtain accurate implant alignment. This follows from considerable literature that supports implant alignment as the most important factor in the long-term success of the prosthesis. To date, however, CAS systems have not been described that would facilitate intraoperative soft-tissue balancing in knee arthroplasty.

Recently, efforts have been made to add soft tissue balancing capability to existing computer assisted knee surgery systems. For example, one group used information from a computer guided system (Orthopilot) to assess and balance ligament tension during total knee replacement (TKR) surgery, as described by Sarin V K and Stulberg D, in an article entitled Abstracts from CAOS International 2001 First Annual Meeting of the International Society for Computer Assisted Orthopaedic Surgery Davos, Switzerland, Feb. 7-10, 2001, Comput Aided Surg, 6(2) 111-130, 2001. The articles concludes that the CAS system is useful in guiding the surgeon in the need for ligament releases and makes it possible to correlate intra-operative stability and flexion with post-operative function.

Articles by Essinger, et al; Mommersteeg, T. J.; Huiskes, R.; Blankevoort, L.; Kooloos, J. G.; and Kauer, J. M., entitled An inverse dynamics modeling approach to determine the restraining function of human knee ligament bundles, J Biomech, 30(2) 139-46., 1997; and by Wilson, D. R.; Feikes, J. D.; Zavatsky, A. B.; and O'Connor, J. J., entitled The components of passive knee movement are coupled to flexion angle, J Biomech, 33 {4) 465-73, 2000 have reported on the passive kinematics of the knee as dictated by the relevant structures. Others articles, including the Chen et al; Martelli et al. articles, describe a kinematic model specific for structures found in the artificial knee. These articles describe the feasibility of a model that assumes the contact condition of the femoral component on the tibial component will be such to minimize the total strain energy stored in the ligaments of the knee. This model purportedly allows prediction of the trajectories of the points of contact between the femoral and tibial prosthetic components as well as the state of strain in the ligaments over the course of flexion. These clinical indications can be valuable for post operatively assessing the performance of the knee for a given component placement.

For a given patient there exist two approaches to proper placement of the prosthetic knee components. The first approach is aimed at satisfying all the necessary requirements for proper alignment with the mechanical axis and any other component specific requirements (these may vary with the manufacturer). This approach may be referred to as optimal placement for alignment. The second approach is aimed at minimizing the strain in the surrounding ligaments throughout the range of motion without regard for proper component alignment. This approach may be referred to as optimal placement for soft tissue balance.

If it were possible to find the optimal placement for soft tissue balance, it could then be compared to the optimal placement for alignment to give an indication of the state of soft tissue balance. It would then be left to the surgeon as to which placement they prefer or if they would prefer a compromise of the two extremes. Given current practices, however, the majority of surgeons would typically select optimal placement for alignment and adjust the soft tissues to bring them as close as possible to a balance. However, given results as reported in articles such as an article by Tew, M., and Waugh, W., entitled Tibiofemoral alignment and the results of knee replacement, Journal of Bone and Joint Surgery—British Volume, 67(4) 551-556, 1985, there may be a trend towards placement for optimal soft tissue balance as this may prove an important condition for long term success.

There remains a need in the art for an intraoperative approach for determining soft tissue balancing with respect to component placement in total knee replacement surgery. In particular, methods and systems are needed to optimize both component alignment and soft tissue balance. More particularly, methods and systems are needed to precisely estimate the attachment sites and lengths of ligaments for a patient and to correlate these with prosthetic component placement so that a surgeon may reliably plan knee replacement surgery to achieve a optimum combination of component alignment and soft tissue balance without need for the trial and error guess-work or post-operative assessments used in the prior art.

### SUMMARY OF THE INVENTION

The present invention provides techniques to quantitatively determine the degree of soft tissue constraints for knee replacement surgery that can be used to plan the surgical procedure prior to making final bone cuts and to optimize component placement parameters for maintaining soft tissue balance of the replacement knee.

One aspect of the invention is a manipulation-based method for quantifying soft tissue constraints in joint replacement surgery. The method includes resecting a proximal segment of a tibia of the subject and providing an initial estimate of an attachment sites (origin and insertions) for each ligament in either a two ligament model that includes the medial collateral and lateral collateral ligaments, or a three ligament model that also includes the posterior cruciate ligament. The tibia is distracted to draw tension on each of the ligaments and while maintaining the tension, the tibia is moved or attempted to be moved in a plurality of different directions relative to the femur. A plurality of displacement positions of the tibia are detected when the tibia is moved in the different directions and the detected displacement positions are represented in a defined coordinate system. A plurality of new estimates of the ligament attachment sites are made by transforming the initial estimate into the defined coordinate system when the tibia is moved to the plurality displacement positions. A plurality of ligament lengths may be calculated from the plurality of estimates of new attachment sites. A final estimate of ligament attachment position and neutral ligament length for the ligaments is then determined by minimizing deviations between the plurality of new estimates of ligament positions and lengths.

Another aspect of the invention is a method for determining placement parameters for the femoral and tibial component of an artificial knee. This aspect includes defining at least one of coordinate systems F_{f} and Fₜ, where F_{f} has an origin representing a point on the femoral component and Fₜ has an origin representing a point on the tibial component. The foregoing estimate of ligament attachment positions and neutral ligament lengths for the medial collateral, lateral collateral and optionally the posterior cruciate ligaments are transformed into the coordinate systems Ff and Ft. An initial estimate of placement parameters for the femoral and tibial components is made. The femoral component placement parameter includes at least one parameter selected from femoral varus/valgus alignment, femoral internal/external alignment, femoral anterior/posterior position and femoral proximal/distal position, and the tibial component placement parameter includes at least one parameter selected form tibial varus/valgus alignment, tibial tilt and tibial proximal/distal position. The tibia is virtually moved in a plurality of flexion angles relative to the femur and for each of the selected flexion angles: strain energy for the at least two ligaments is calculated; a position of the tibial component relative to the femoral component that minimizes a total strain energy comprised of the sum of the strain energies on the ligaments is determined; If at least one of the ligament lengths at this position is less than the neutral length (i.e., is slack), a second adjustment is made to identify the position of the tibial component relative to the femoral component that maximizes the sum of slacknesses of the slack ligaments at the given flexion angle; and a first sum of deviations for the ligament lengths is determined, the first sum being the sum of deviations from the neutral ligament lengths for each of ligament when the tibial component is positioned relative to the femoral component to minimize the total strain energy The strain energy may include both true strain energy, corresponding to ligaments being under tension, and pseudostrain energy, corresponding to ligaments being slack; deviations from neutrality may be weighted differently for the two conditions, according to the surgeon's preference. A total ligament deviation comprised of a sum of the first sum of ligament deviations for all the flexion angles is then determined. Final placement parameters for the components are determined by calculating positions for the prosthetic components that minimize the total ligament deviation.

Each of the foregoing aspects of the invention are typically implemented using a CAS system configured with instructions for executing the acts of positional detection and the minimizing calculations required for the methods above. Accordingly, in another aspect, the invention includes CAS systems configured to accomplish the foregoing methods.

### BRIEF DESCRIPTION OF THE DRAWINGS

FIGS. 1A and 1B graphically illustrate soft tissue balance and imbalance, which are addressed by the present invention.

FIGS. 2A and 2B graphically illustrate adjusting flexion gap according to methods of the prior art.

FIG. 3 illustrates a block model of bone and ligament coordinate frames used in one aspect of the invention.

FIG. 4 is a flowchart outlining a manipulation-based method for identifying ligament attachment sites according to one aspect of the invention.

FIG. 5 is a photograph illustrating a testing setup according to one aspect of the invention.

FIGS. 6A and 6B are charts showing low intraoperator error in a two and three ligament model, respectively, according to an aspect of the invention.

FIGS. 7A and 7B are charts showing interoperator differences in estimated attachment sites and other ligament parameters in the two and three ligament model, respectively.

FIG. 8 depicts a plurality of estimated ligament attachment sites obtained from moving the tibia in plurality of different directions.

FIG. 9 is a block diagram illustrating a method of optimizing component placement parameters according to another aspect of the invention.

FIG. 10A illustrates coordinate system for the tibia, femur and knee joint component according to one embodiment of the invention. FIG. 10B illustrates a corresponding knee model used for determining component placement parameters.

FIG. 11 shows passive kinematics of the femoral component with respect to the tibial component before and after using the placement algorithm according to the method of the invention.

FIG. 12 shows variances in placement parameters determined from the thirty trials on porcine specimens using the methods of the invention.

FIG. 13A shows variance in deviation from neutral length of each ligament for over 30 component placements determined using the methods of the invention. FIG. 14b shows variance in kinematics predicted by a passive kinematic model for the over 30 placements.

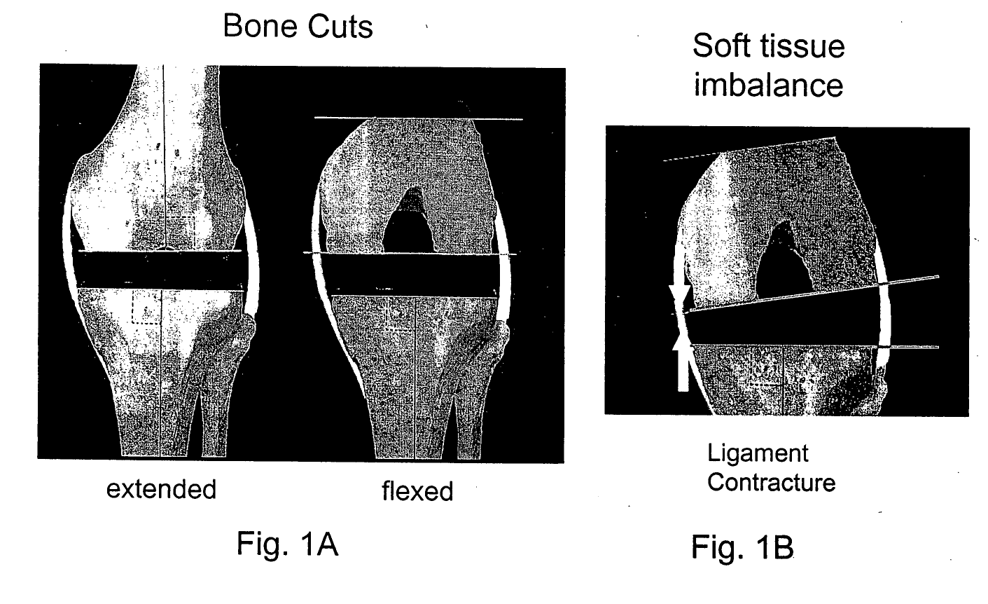

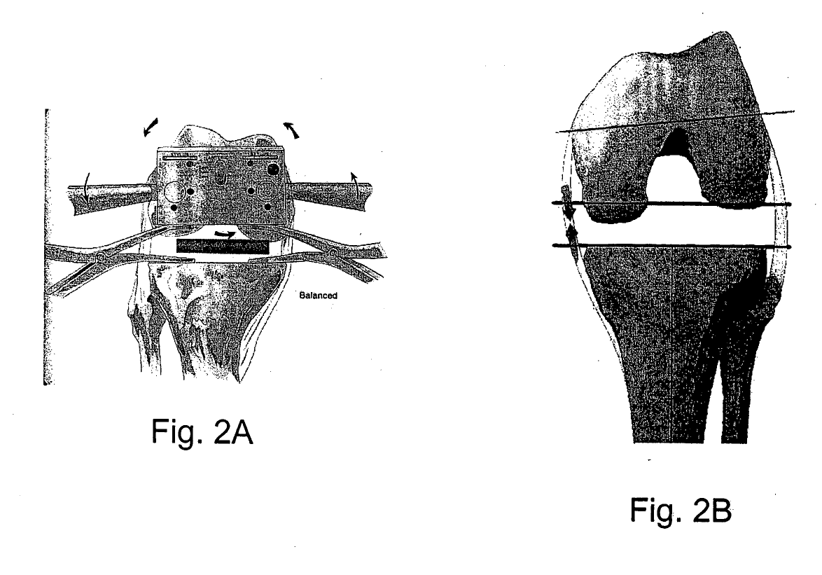

### DETAILED DESCRIPTION OF THE INVENTION

In the description that follows, citation is made to various references that provide information that may assist one of ordinary skill in the art to better understand and/or practice the invention. Each such reference contains information that is readily accessible and/or is well known to one of one of ordinary skill in the art and is incorporated herein by reference to the extent that may be needed to facilitate practice of the invention. In addition, although the description that follows describes the invention in the context of knee arthroscopy, one of ordinary skill in the art will recognize that the practice of the invention is not limited to knee replacement, but is applicable to surgical procedures with any joint where prosthetic components need to be aligned with respect to bones while maintaining a soft tissue balance in attached ligaments.

### Assessing Soft-Tissue Constraints

According to one embodiment of the invention, soft tissue constraints are intraoperatively assessed by determining the functional attachment sites and neutral lengths (\overline{L}) of the ligaments surrounding the knee. A resected tibia is manipulated in a plurality of orientations with respect to the femur and measurements are made of the relative position of the tibia with respect to the knee at the plurality of orientations. Motion data is captured while manually distracting and manipulating the knee to determine the effective ligament attachment sites and lengths. In an advantageous embodiment, the measurements may be made prior to any femoral bone cuts, which thereby allows for planning of the remaining portions of the procedure in manner to optimize effective tissue balance and prosthetic component placement. Through mechanical manipulation of the tibia with respect to the femur, the effective constraints introduced by the soft tissue may be accurately quantified. Alternatively, the first cut may be made on the femur and the tibia left unresected. Further, it may be possible to achieve a sufficient degree of manipulation without making any bone cuts, having simply removed at least one of the menisci, any osteophytes and any other extraneous soft tissue that will not be retained after implanting the components.

In an exemplary embodiment, the surgeon first makes the standard tibial plateau cut (if so desired, this cut can be conservative, leaving enough bone stock for a further trim cut to adjust the final location of the cut). In one embodiment, only the tibial cut need be made to practice the invention. In another embodiments, a femoral bone cut may also be made, although such an additional cut is not needed and may not be preferred in most practices of the invention. Once the proximal tibial segment is removed, the surgeon distracts the tibia until all ligaments are tensed, then attempts to manipulate the tibia in all possible directions and orientations, some of which will be resisted by the tensed ligaments. The ligaments are mathematically modeled as inextensible strings or as multifibre bundles where data for describing the behavior of multifibre bundles is available and an optimization routine is executed to identify the effective attachment sites (origins and insertions) and lengths of the ligaments.

Optimization Routine. An optimization algorithm based on a model representation of the knee is used to determine the ligament attachment sites and lengths. At least two ligaments are assessed in the model. In one embodiment, a two-ligament model consisting of only the medial collateral (MCL) and lateral collateral (LCL) ligaments is used. In another embodiment, a three-ligament model consisting of the two collateral ligaments and the posterior cruciate ligament (PCL) may be used. These two models represent the most common situations in total knee arthroscopy (TKA), which include that of a PCL sacrificing or substituting implant (where the PCL is resected) and a PCL retaining implant.

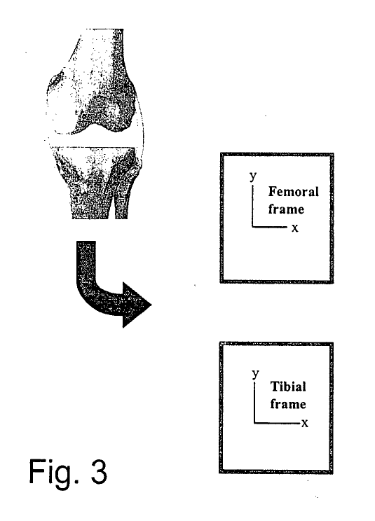

The two bones and ligaments are modeled as two blocks and inextensible strings, which are graphically depicted in FIG. 3. The tibia and the femur are each assigned a unique reference frame (coordinate systems F_{T} and F_{F}, respectively) each which may be defined arbitrarily by a marker array attached to the bone. An initial estimate is made for the ligament attachment sites relative to the reference frames by using a subjective or semi-subjective guess of ligament position such as is ordinarily made by a typical practitioner of ordinary skill in the art, for example, by palpitation. In a typical embodiment, the position of the estimate is indicated by placing a stylus with a light emitting diode at the estimated position and detecting the position of the light emitting diode using an optoelectronic detection and input device commonly available with CAS systems. The origins of ligament attachment sites are represented in the femoral frame F_{F} and the insertions of ligament attachment sites are represented in the tibial frame F_{T}.

An example optoelectronic metrology system suitable to collect data for the practice of the invention is Flashpoint 5000, Inage Guided Technologies (Boulder Co.). Marker arrays comprised of three infrared light emitting diodes are rigidly attached to each of the femur and tibia using bone pins. The two marker arrays are used to define the two separate Cartesian coordinate systems F_{F} and F_{T} having an origin rigidly fixed on a point of the respective bones for the femoral frame and tibial frame, respectively. A foot pedal or other suitable activation device is used activate the data collection system. The positions of the markers are captured in “displacement mode” for the Flashpoint 5000, which captures a new data point when the tibial markers have moved 2.5 mm in space.

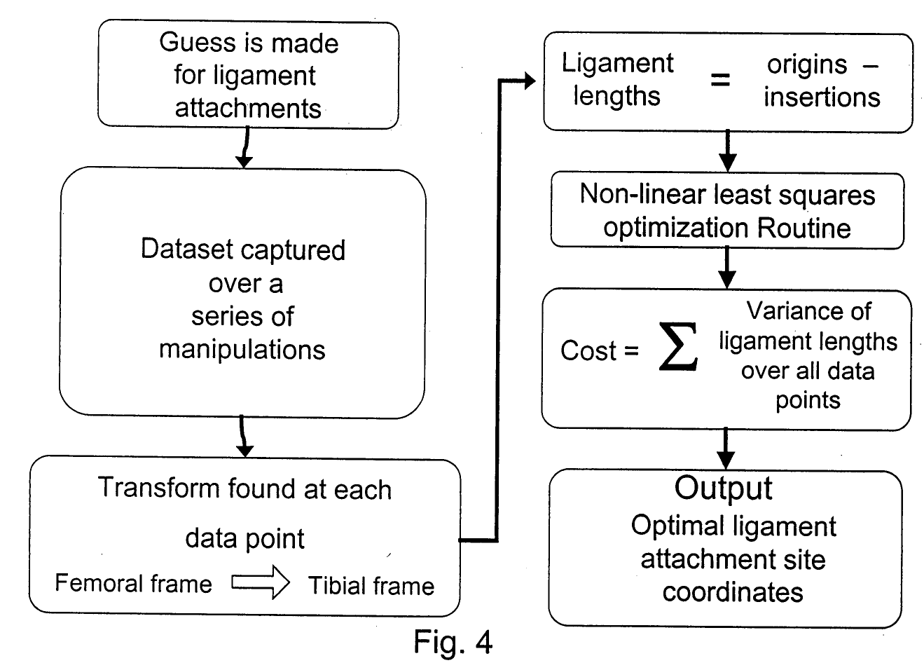

FIG. 4 shows a schematic overview of the optimization procedure used in this embodiment of the invention. After the initial estimate is made, tension is drawn on the ligaments by distracting the tibia, which is then manipulated in a plurality of different directions while maintaining the ligaments under tension. In a typical practice, a marker array such as pins having light emitting diodes are firmly attached to the tibia and the femur and the position and orientation of the tibia with respect to the femur. A homogeneous transform relating the femoral frame to the tibial frame (or vice versa) is used to specify the transformation of positional coordinates between frames. The coordinates of the ligament origins are represented in the femoral frame F_{F} and the insertion sites are represented in the tibial frame F_{T}. and the lengths of the ligaments found by simple subtraction (e.g., position of origin minus position of insertion). The position of the tibial frame in the femoral frame is captured over the plurality of different positions in space while maintaining tension in the ligaments.

As the tibia is moved from position to position, the location of the estimated attachment sites relative to the tibia and/or the femur will change in the respective coordinate systems F_{T} and F_{F}. At each position, new estimates of the position of the ligament attachment sites (and lengths) are determined by transforming the initial estimate into the respective coordinate system when the tibia is moved to the different positions. A non-linear least squares optimization algorithm (e.g., the trust region-reflective algorithm) is used to determine the insertion and origin locations of ligaments that minimize the variance of ligament lengths over the entire dataset. Thus, the inputs to the optimization procedure are the initial estimate of the ligament origin and insertion sites and the detected positions of relative displacement of the tibia. Subsequent estimations for the ligament attachment sites are made by detecting the position of the tibia at each of the plurality of positions to which the tibia is moved and a data point is taken and transforming the initial estimated position of the attachment sites into the tibial frame and femoral frame at each of the plurality of positions. The output is an optimized set of positions for the x, y, z coordinates of ligament attachment sites that minimizes the change in ligament lengths over the entire data set. The optimized attachment sites are used to calculate ligament lengths at each data point and the resulting coordinates for the attachment sites and lengths are calculated and reported as the coordinates that minimize the deviations between the estimated lengths.

### EXAMPLE I

### Determination of Ligament Length and Attachment Sites with Porcine Subjects

The Flashpoint 5000 system has a typical accuracy of approximately 0.5 mm in tracking infrared emitting diodes (IREDs) within a 1 m diameter volume. The noise of the system was determined from the data collected from one dataset from one trial. The position of the emitters attached to the tibial array was used to construct a tibial reference frame. A transform was then found from the femoral frame into the tibial frame and the tibial emitter positions were transformed into the tibial frame. This was repeated for all data points in the set and the error in the emitter locations calculated. The error was determined to be 0.2 mm SD for typical data sets. A perfect data set was generated using Working Model 3D© version 3.0 (Working Model Inc., 1996) with a model of similar geometry as the test specimens. White noise with zero mean and 0.2 mm SD was added to the generated dataset to represent the measurement error of the Flashpoint 5000 system. Thirty datasets with random noise were generated to assess the variability in the optimization output due to measurement error. Each model was tested for a full 0-90 degree range of motion and a smaller 0-30 degree range of motion.

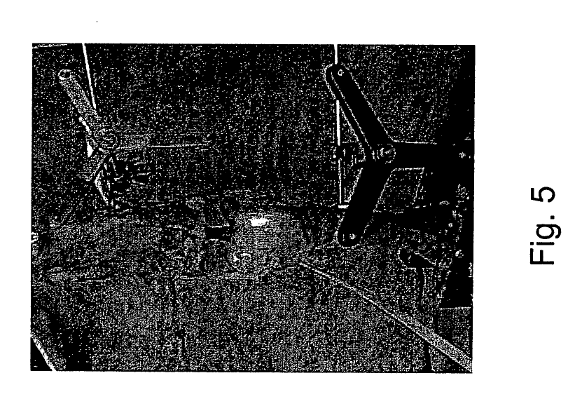

Specimen Preparation and Limb Manipulations. Six fresh-frozen intact porcine hind limbs were obtained in accordance to the University of British Columbia animal testing regulations. The animals were six months old and had a mean weight of 150 kg. Prior to testing the limbs were allowed to thaw for a period of 10-12 hours. Each limb was dissected leaving only the tibia, femur and knee capsule intact. The patella and patellar ligament were then resected along with the posterior capsule. The menisci were then resected as well as the anterior cruciate ligament as is performed in most knee arthroplasties. All other structures were removed leaving only the MCL, LCL and PCL intact. The PCL was removed for the two ligament specimens. Using an appropriate cutting guide (e.g., Depuy Inc.) and oscillating saw, the proximal end of the tibia was cut in accordance to the manufacturers recommendations (e.g., Johnson and Johnson, Inc.) and is depicted in FIG. 5.

The proximal end of the femur was then rigidly mounted to a tabletop to represent an intact hip joint. A small cord was attached to the distal end of the tibia to allow the user to effectively grasp the limb. The limb was distracted manually by applying tension on the tibia in the distal direction. Care was taking to maintain distraction at a level greater than 20 lbs throughout the data capture. The limb was then manipulated in seven distinct motions to explore all the potential degrees of freedom of the two bones and observe the constraint provided by the ligaments. These motions were as follows:
i. Anterior/Posterior manipulation
ii. Medial/Lateral manipulation
iii. Flexion/Extension manipulation about a line connecting ligament origin sites
iv. Internal/External rotation
v. Flexion/Extension about a line connecting ligament insertion sites
vi. Varus/Valgus rotation
vii. Straight distraction

While it is preferable to manipulate the tibia in each of the aforementioned directions to achieve as many data points as practical, the invention can be practiced by manipulation of the tibia in at least two, at least three, at least four, at least five or at least six, or at least of the seven directions. In most practices, the tibia should be manipulated in a number of directions equal to at least 6 minus the number of ligaments remaining. In a typical practice, the manipulation will be in seven directions to ensure that rotations around the mediolateral axes at both the insertions and origins of the collateral ligaments are significant, although strictly speaking only one of the manipulations of Flexion/Extension about the origin or insertion sites is necessary.

For all the motions, care was taken to ensure that all ligaments were taut throughout the motion. Some motions were unable to be completed as a result of the constraint introduced by the ligaments. For example, varus/valgus rotation was not possible to complete without one or more of the ligaments going slack.

The effect of different fiber bundles being active at different flexion angles was explored by performing the seven manipulations about three distinct flexion angles. The seven motions were first performed about the full extension position (0 degrees of flexion) with the flexion/extension motion limited to the first 30 degrees of flexion. The seven manipulations were then performed about the full flexion position (90 degrees of flexion) with the flexion/extension motion limited to between 60 and 90 degrees. The seven motions were finally performed about the mid flexion position (45 degrees of flexion) with the flexion/extension motion limited to between 30 and b0 degrees. These three separate datasets were considered one trial.

Experimental Protocol. Three operators were recruited for this experiment. The six specimens were divided into two groups. Three were prepared with MCL, LCL and PCL ligaments intact (three-ligament model) and three were prepared with only the MCL and LCL intact (two ligament model). Each of the three operators performed 30 trials each on a single specimen from each group. This data was collected for analysis of inter-operator variability. One of the operators performed 30 trials on each of the remaining four specimens to determine the inter-specimen variability. Data from a single user on a single specimen (30 trials) was used to determine intra-operator variability.

Each operator digitized a guess for the origins and insertions of each ligament using a stylus once per specimen. After all trials were performed on a specimen, the ligaments were dissected from the bones and the perimeter of their anatomical attachment sites digitized. Approximations of the hip and ankle centers were also digitized. The digitized anatomy was used to construct reference frames at the center of the distal femur and proximal tibia. These frames were used to convert the optimization parameters to values expressed in terms of common anatomical references.

Data analysis. The output of the measurement protocol resulted in 21 values for the three-ligament model (18 coordinates and 3 ligament lengths) for a single set of data and 14 values for the two-ligament model (12 coordinates and 2 ligament lengths). This corresponds to three coordinate positions x, y, z for each ligament for each of the origin and insertion attachment sites and the lengths of the ligaments. Each trial consisted of three datasets; one centered at each of the distinct flexion angles. A fourth dataset was constructed by combining the three datasets for a single trial. The variance in each of the 21 variables was calculated for each dataset over the 30 trials. For comparison of the difference in ligament attachments and lengths due to the motion about distinct flexion angles (effect of fiber bundles), the 95% confidence interval for the difference of the means of each parameter was computed. The 95% confidence interval for the difference in the 21 or 14 parameters was also computed to compare each of the three operators. For the inter-specimen comparison, the variance of each of the parameters for all three specimens was compared as the means could not be directly compared due to the difference in reference frame locations across the specimens.

Results, Model Validation. The measurement errors introduced in the simulation affected the output of repeated optimizations. The average error was 0.1 mm SD for identification of ligament attachment sites and 0.1 mm SD for overall ligament length for the full range of motion model. The errors increased significantly to 1.3 mm SD and 1.0 mm SD for the reduced range of motion model. The measurement errors were seen to have a much large effect for the smaller manipulations (30 degrees of flexion.) For the three ligament model, the average error was 0.3 mm SD for identification of ligament attachment sites and 0.6 mm SD for overall ligament length for the full range of motion model. Again, the errors increased significantly to 2.3 mm SD and 0.7 mm SD for the reduced range of motion model.

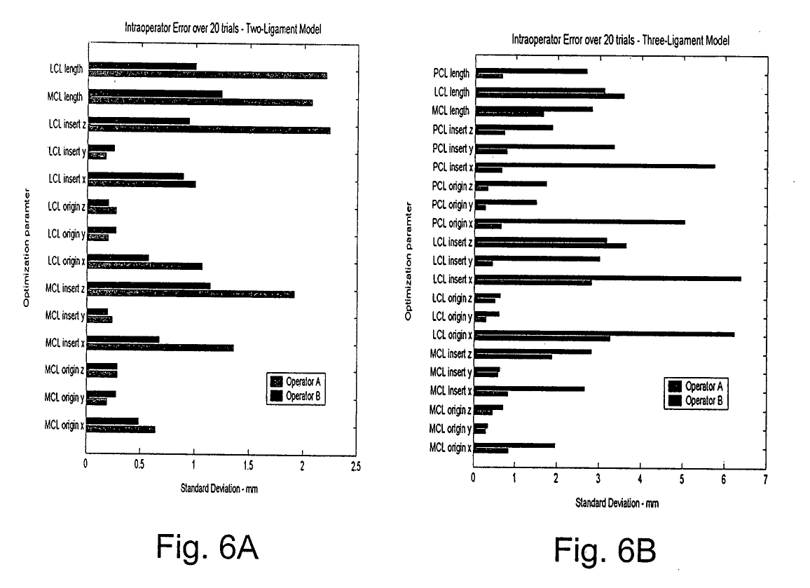

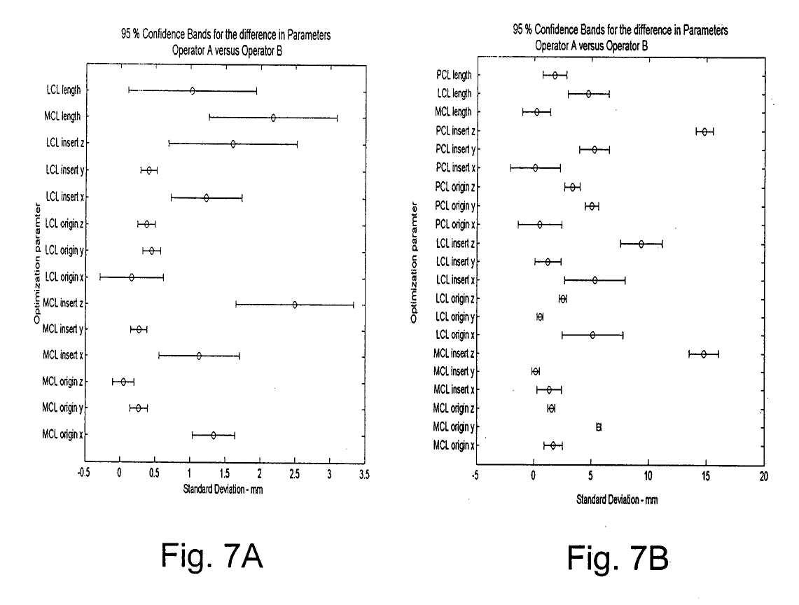

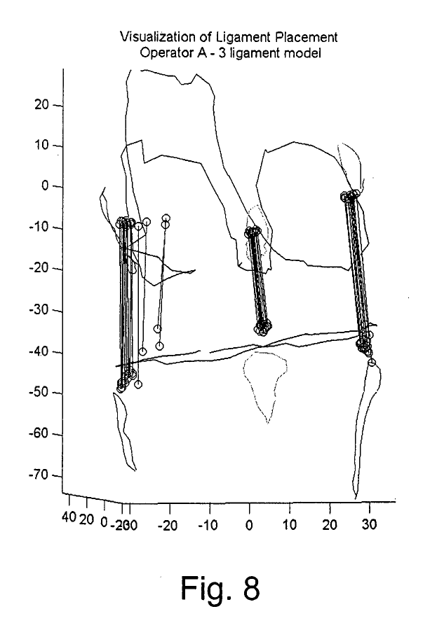

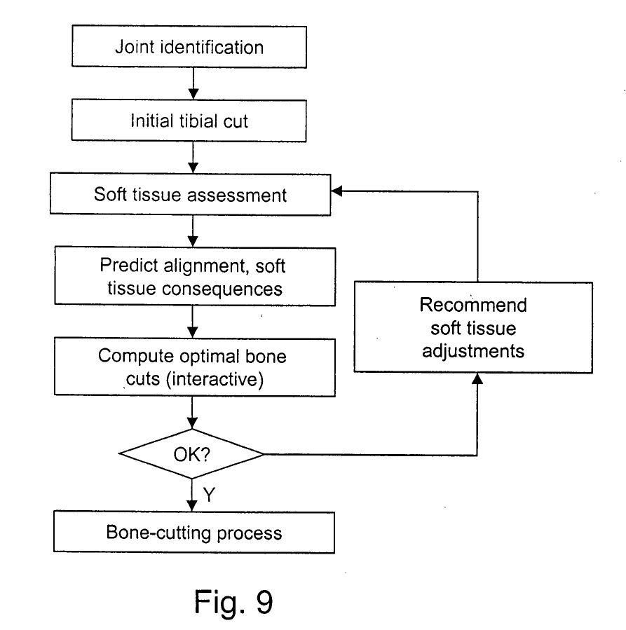

Result Repeatability. FIGS. 6 and 7 show the overall repeatability of the procedure. Intraoperator repeatability is on the order of 0.5-1 mm and 2 mm for the two and three ligament models respectively, while interoperator repeatability is somewhat larger at 1 and 4 mm, respectively. FIG. 8 shows that the locations of the estimated ligament attachment sites are in close proximity to the digitized ligament origins and insertions. Hence, origins, insertions and lengths of the ligaments can be identified with very good intraoperator repeatability (on the order of 1-2 mm) and reasonable interoperator repeatability (2-3 mm). These repeatability values are sufficiently good that, if obtained in live surgery, the soft tissue quantification technique should be of value in total knee replacement surgery.

### Determining Parameters for Component Placement

Other aspects of the invention include methods and systems for properly positioning prosthetic components in knee replacement surgery, preferably by using the previously described methods and systems for determining the location of ligament attachment sites and lengths of ligaments. According to one embodiment of the invention, prosthetic components are positioned using a passive knee kinematic model of the knee, and using a series of instantaneous quasi-static solutions to energy minimization, such as described for example in the previously cited article by Chen et al, which is incorporated herein by reference. In addition, extension or slack in ligaments as a function of flexion angle may be determined from a quasi-static model and used with the knee kinematic model in combination with representations of positional coordinates for various test positions of prosthetic components. A component placement that results in the optimal ligament behavior is then calculated to assist the surgeon in planning and executing the knee replacement surgery.

In one embodiment, simplified geometries are used to represent the prosthetic components, although methods exist for handling more complex and realistic geometries (for example, Chen et al). For example, the femoral component may be represented by a cylinder and a flat plate may be used to represents the tibial component. Line contact between the cylinder and flat plate is assumed to occur at all times. Current prosthesis designs have bearing surfaces that are not geometrically congruent, which introduce additional degrees of freedom in knee motion that are captured by the represented geometries. The aforementioned representations of component geometries are therefore merely simplified examples of many possible representations that one of ordinary skill in the art might use in the methods of the present invention. In particular, one would normally use an accurate model of the components that the surgeon intends to implant.

The coordinate systems used for representing positions of components are selected to be compatible with typical CAS systems available in the art, for example, the prototype system available at the University of British Columbia medical center or others such have been described, for example, by Martelli et al, which is incorporated herein by reference. For representing positions of the major bones two Cartesian coordinate systems are defined for the major bones of the lower limbs analogously to the reference frames used to capture positional data for the plurality of tibia positions described above. In one embodiment, and for convenience only, the z axes are directed along the mechanical axis of the bone with the proximal direction being positive. The x-axes are perpendicular to the z-axis directed positive to the right in the coronal plane. The y axes are determined from the relationship y=z X x, which results in positive being in the anterior direction. The origin of the coordinate system for the femoral frame (F_{F}) is located at the midpoint of the origins of the two collateral ligaments (i.e., lying on the transepicondylar axis, the primary flexion axis, as described by Grood and Suntay, which are incorporated herein by reference). The origin of the tibial frame (F_{T}) is located at the midpoint of the insertion sites of the collateral ligaments and is considered fixed in space. The coronal plane for each bone is separately defined as the plane made up of the two ligament attachment points, and the center of the femoral head or the ankle center (defined as the midpoint between the maleoli.)

For representing the positions of the prosthetic components, two additional Cartesian coordinate systems are defined for the components. The femoral component frame (F_{f}) is positioned at the center of mass of the cylinder with the x-axis directed along the major axis of the cylinder. The z-axis was perpendicular to the x-axis with the y axis defined as y=z X x. The tibial component frame (Fₜ) is positioned on the proximal surface of the flat plate. The z-axis is coincident with the normal of the flat plate, positive directed away from the center of the plate. The x and y axis are located in the plane of the flat plate forming a right hand coordinate system with the z-axis.

Coordinate system transformations. As mentioned above, the component placement model uses as an input the defined ligament positions and neutral ligament lengths \overline{L} obtained as previously described herein. To calculate the ligament lengths, it is necessary to derive a homogeneous rigid-body transform from the femoral frame to the tibial frame (T_{TF}). Homogenous rigid body transforms are described, for example, in a textbook by Sciavicco, L., and Siciliano, B., entitled Modeling and control of robot manipulators, Edited, xvii, 358, New York, McGraw-Hill Companies Inc., 1996, which is incorporated herein by reference. In one embodiment, the homogenous rigid body transform is found by multiplying the successive transforms as follows, moving from the femur to the tibia:
T_{TF}=T_{Tt}*T_{tf}*T_{fF} (1)
where:
T_{Tt}=transform from Fₜ to F_{T}
T_{tf}=transform from F_{f} to Fₜ
T_{tF}=transform from F_{F} to F_{f}

The pose (i.e., the positional orientation) of each prosthetic component with respect to the bones is represented by the homogeneous transform between the two associated frames. The homogeneous transform is made up of basic fixed frame rotations and displacements as described by Sciavicco, et al. A basic translation along the current axes a distance a in the x direction, b in the y direction and c in the z direction is represented by:

$${Trans}_{x , a : y , b : z , c} = [ \begin{matrix}1 & 0 & 0 & a \\ 0 & 1 & 0 & b \\ 0 & 0 & 1 & c \\ 0 & 0 & 0 & 1\end{matrix} ] \qquad ( 2 )$$

Rotation about the current x axis, an amount α:

$${Rot}_{x , α} = [ \begin{matrix}1 & 0 & 0 & 0 \\ 0 & cos α & - sin α & 0 \\ 0 & sin α & cos α & 0 \\ 0 & 0 & 0 & 1\end{matrix} ] \qquad ( 3 )$$

Rotation about the current y axis, an amount φ:

$$Rot y ϕ = [ \begin{matrix}cos ϕ & 0 & sin ϕ & 0 \\ 0 & 1 & 0 & 0 \\ - sin ϕ & 0 & cos ϕ & 0 \\ 0 & 0 & 0 & 1\end{matrix} ] \qquad ( 4 )$$

Rotation about the current z axis, an amount γ:

$${Rot}_{z γ} = [ \begin{matrix}cos γ & - sin γ & 0 & 0 \\ sin γ & cos γ & 0 & 0 \\ 0 & 0 & 1 & 0 \\ 0 & 0 & 0 & 1\end{matrix} ] \qquad ( 5 )$$

The transform T_{fF} represents the position of the femoral frame in the femoral component frame and was defined by four parameters:
Femoral varus/valgus alignment (VV_{F})=rotation about y-axis of F_{f}
Femoral internal/external alignment (IE_{F})=rotation about z-axis of F_{f}
Femoral anterior/posterior position (AP_{F})=translation along y-axis of F_{f}
Femoral proximal/distal position (PD_{F})=translation along z-axis of F_{f}

The transform T_{Tt} represents the position of the tibial component in the tibial frame and was defined by three additional parameters:
Tibial varus/valgus alignment (VV_{T})=rotation about y-axis of F_{T}
Tibial component tilt (Tilt_{T})=rotation about x-axis of F_{T}
Tibial proximal/distal position (PD_{T})=translation along z-axis of F_{T}

These seven parameters are the only placement parameters for the components of a prosthetic joint that can be modified to affect knee kinematics in the current model, due to its innate simplicity. When using more realistic models of the implant components, additional parameters describing flexion/extension and mediolateral positioning of the femoral component and internal external rotation, anterior/posterior translation and mediolateral translation of the tibial component may be required. In addition, the size of the components may be treated as a design variable.

The two transforms are calculated using fixed frame transformations with the actual transformations occurring in the reverse order in which they are multiplied:
T_{fF}=Rot_{y}(VV_{F})*Rot_{z}(IE_{F})*Trans_{x,y,z}(0,AP_{F},PD_{F}) (6)
T_{Tt}=Trans_{x,y,z}(0,0,PD_{T})*Rotₓ(Tilt_{T})*Rot_{y}(VV_{T}) (7)

The orientation of the femoral component with respect to the tibial component can be described by a homogeneous transformation derived from five parameters:
I. Anterior/posterior displacement (AP_{comp})=translation along y-axis of Fₜ
II. Medial/lateral displacement (ML_{comp})=translation along x-axis of Fₜ
III. Proxial/distal displacement (PD_{comp})=translation along z-axis of Fₜ
IV. Internal/external rotation (IE_{comp})=rotation about z-axis of Fₜ
V. Flexion/extension rotation (FE_{comp})=rotation about x-axis of Fₜ
   Thus the transform T_{tf} is calculated as follows:
   T_{tf}=Trans_{x,y,z}(ML_{comp},AP_{comp},PD_{comp})*Rot_{z}(IE_{comp})*Rotₓ(FE_{comp}) (8)

Assuming that the components are always in contact, and that the femoral component is a cylinder, PD_{comp} is a constant equal to the component radius. The flexion angle is set to a distinct value for evaluation in this model.

Passive knee kinematics. As previously mentioned, the main ligaments of the knee are the anterior cruciate ligament (ACL), the posterior cruciate ligament (PCL), the medial collateral ligament (MCL) and the lateral collateral ligament (LCL), and during implantation of total knee prostheses the ACL is resected, and therefore not relevant to this model. The origins of the three remaining ligaments are represented as x,y,z Cartesian coordinates in F_{F}, with the insertion locations represented in F_{T}. A fixed component placement is assumed for the femoral and tibial components (T_{fF} and T_{Tt}).

For a distinct flexion angle (FE_{comp}) and an initial estimate for the component placement parameter AP_{comp}, ML_{comp} and IE_{comp}, the transformation T_{TF} is found. Using this transformation, the locations of the ligament origins obtained according to the previously methods are determined in F_{T}. The length of each ligament is defined as equal to the difference between its origin and insertion locations. Ligaments are modeled as tension-only linear springs, with the strain energy increasing quadratically with extension and being zero in compression. The total energy of the system is defined as the sum of the strain energies of the individual ligaments. Let Lᵢ be the instantaneous length of the i^{th} ligament, \overline{L}ᵢ its neutral length and Kᵢ be its spring constant. In a typical practice, \overline{L}ᵢ is an estimate obtained from the tibial distraction method described herein before. The strain energy of each ligament is defined as:

$${E}_{i} \{ \begin{matrix}\frac{{{K}_{i} ( {L}_{i} - {\overline{L}}_{i} )}^{2}}{{\overline{L}}_{i}^{2}} & if {L}_{i} \geq {\overline{L}}_{i} \\ 0 & if {L}_{i} \leq {\overline{L}}_{t}\end{matrix} \} i = MCL , LCL , PCL \qquad ( 9 )$$

The total strain energy is defined as:
E_{Total}=E_{MCL}+E_{LCL}+E_{PCL} (10)

The parameters AP_{comp}, ML_{comp}, and IE_{comp} are found such that this stain energy is at a minimum using a conventional non-linear unconstrained optimization algorithm (e.g., Quasi-Newton) at distinct flexion angles in the range of 0°-135°. Thus the input variable of the passive kinematics algorithm is the flexion angle and the outputs are the three orientation parameters AP_{comp}, ML_{comp}, and IE_{comp} that define the relative position of the tibial and femoral components.

Slack and the component placement algorithm. The objective of the component placement algorithm is to determine the seven placement parameters for the components of the prosthetic joint that will result in the ligaments lengths remaining at their neutral lengths throughout the range of 0°-135° flexion. Thus, a placement is to be found which minimizes not only the stretch in the ligaments, but also the slack in the ligaments.

The passive kinematic model described above is preferably used to observe the stretch in ligaments throughout the range of motion for a given component placement. However, this model is unable to quantify the amount of slack resulting from a component placement because it is possible for one or more ligaments to be slack at the energy minimum, resulting in multiple solutions for this optimization. To penalize this component placement, it is then necessary to compute the position, subject to having the same or less stored energy, that results in the most slackness in the ligaments. This is found by minimizing the sum of the lengths of each ligament, subject to the energy being less than or equal to that found by the passive kinematic routine:
Total ligament length=Length_{MCL}+Length_{LCL}+Length_{PCL} (11)
The cost associated with this component placement is the deviation of each ligament from its neutral length:
Deviation_{at flexion angle}=|L_{MCL}−\overline{L_{MCL}}|+|L_{LCL}−\overline{L_{LCL}}|+|L_{PCL}−\overline{L_{PCL}}| (12)
In principle, one could weight the different ligaments differently (e.g., in proportion to their cross-sectional area) and could also weight strain and laxity differently, according to the desires of the surgeon.

The steps used in the placement algorithm may be summarized as follows:
1. An estimate is made for the seven component placement parameters.
2. For a distinct flexion angle, the position (AP_{comp}, ML_{comp}, IE_{comp}) that minimizes the strain energy in the ligaments is found.
3. The transform T_{tf} is calculated and the ligament origins are transformed into Fₜ.
4. Each ligament is tested to see if it is in a slack condition (Lᵢ<=\overline{L}i).
5. If one or more of the ligaments is found to be slack, the position that results in the most slack in the ligaments is found, subject to having less than or equal to the strain energy found in step 2.
6. The sum of all three ligament deviations is computed for the given flexion angle using equation 12.
7. Steps 2-6 are repeated for the entire range of flexion angles.
8. The total ligament deviation for this component placement is computed as the sum of deviation in ligament lengths at each flexion angle.
9. Using a conventional non-linear unconstrained optimization procedure (e.g., Nelder-Mead Simplex Method), the seven placement parameters are found that minimize the total ligament deviation.

### EXAMPLE II

### Component Placement Model

A dynamic mechanical model was created using the software package Working Model 3D©™ (Working Model Inc.) to validate the method described herein. The dynamic model consisted of two rectangular blocks, a 25 mm cylinder and a flat plate representing the two bones, femoral component and tibial component, respectively. Ligaments were represented by spring/damper constraints with the spring constants set to zero in compression. The spring attachment points were set to approximate anatomical locations, however for simplicity the collateral ligaments were taken to be symmetric about the sagittal plane. The prosthetic components were virtually implanted with the femoral component centered about the collateral origins.

The passive kinematic model was validated first. The femoral component was set at a distinct flexion angle and was virtually released, coming to rest on the tibial component at the equilibrium defined by the attached springs. Contact between the two components was enforced. This was repeated for the distinct angles in the range of 0°-135°. The resulting orientations were compared to the passive kinematic model.

The component placement algorithm was validated by altering the neutral lengths of the attached ligaments. The lengths of the ligaments over the range of flexion angles were first noted for a standard component placement using the passive kinematic model. The optimal component placement was then found, and the lengths of the ligaments recalculated for comparison.

The degree to which the algorithm is affected by variance in the input parameters was investigated by running the algorithm on a set of 20 ligament location solutions. The variance of the resulting set of 20 solutions for the component parameters was then determined.

Results. To demonstrate the ability of this algorithm to substantially correct a variety of ligament imbalances, the optimization process was simulated using an idealized knee model which is topologically identical to knee prostheses used clinically, but with simpler geometry to facilitate the computations related to parameterizing the constrained subspace, P.

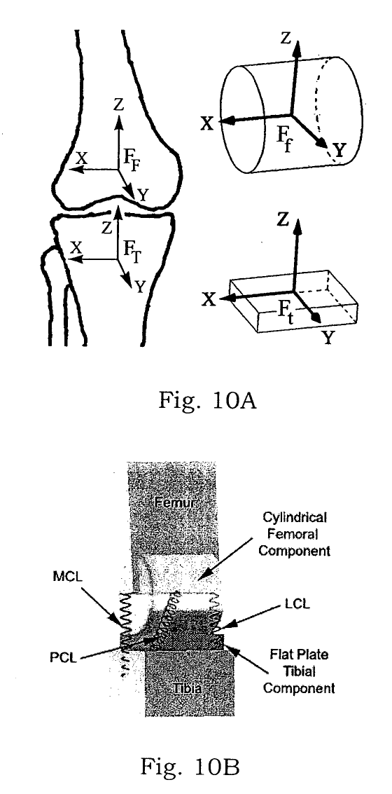

The idealized knee model used is shown in FIG. 10 and consisted of a flat plate to represent the tibial component and a cylinder with a 25 mm radius to represent the femoral component (because the tibial plate is flat, this is equivalent to using two spheres to represent the femoral component, which would produce two contact points on the tibial plate. This is topologically equivalent to unconstrained posterior-cruciate-retaining knee prostheses which also have two contact points with the tibial plate). The coordinates used for the ligament attachment sites are shown in Table 1, and the ligament neutral lengths and their relative stiffness are shown in Table 2. In this model, the stiffness of the PCL is four times that of the collateral ligaments, reflecting the relative cross-sectional area of the ligaments as described in the above-cited article by Martelli, et al. For simplicity in interpreting the results, the MCL and LCL were defined to be mirror images of one another across the sagittal plane through the center of the knee; although more realistic ligament attachment sites easily can be determined. The attachment sites of the PCL were chosen to approximate the action of the PCL in the normal knee.

TABLE 1
Ligament Attachment Site Coordinates for Validation Model

| Ligament | | | | |
|---|---|---|---|---|
| Attachment | Frame | X coordinate | Y coordinate | Z coordinate |
| MCL origin | F_{F} | 30 mm | 0 mm | 0 mm |
| LCL origin | F_{F} | −30 mm | 0 mm | 0 mm |
| PCL origin | F_{F} | 0 mm | 5 mm | −5 mm |
| MCL insertion | F_{T} | 30 mm | 0 mm | 5 mm |
| LCL insertion | F_{T} | −30 mm | 0 mm | 5 mm |
| PCL insertion | F_{T} | 0 mm | −25 mm | 25 mm |

TABLE 2
Ligament Data for Validation Model

| Ligament | MCL | LCL | PCL |
|---|---|---|---|
| Neutral Length | 45 mm | 45 mm | 33 mm |
| Relative Stiffness | 1 | 1 | 4 |

In this nominally correct configuration (corresponding to correct mechanical axis alignment), the ligaments are not necessarily isometric throughout the range of motion, and the placement algorithm was run to predict the placement for optimal balance. The neutral lengths of the ligaments was altered to simulate various ligament imbalances. In the first imbalanced simulation, the MCL was shortened by 5 mm to represent a varus imbalance. In the second simulation, the PCL was shortened by 5 mm to represent a flexion contracture. The MCL and PCL were then both simultaneously shortened to represent a more complex imbalance. A simulation was also performed with the MCL lengthened by 5 mm to investigate the ability of the model to manage a slack ligament. For all simulations, the ligament strain profiles and the kinematics of the knee were calculated both before and after the placement optimization.

The degree to which the placement predicted by the algorithm is affected by variance in measurement of the ligament attachment sites was investigated by running the placement optimization on a set of 30 ligament attachment and neutral length solutions from Example I. In that Example, the attachment sites estimates had an average standard deviation of 0.9 mm for ligament locations and 1.1 mm for ligament neutral lengths.

Table 3 presents the component parameters resulting in optimal placement for soft tissues as found by the placement algorithm for all simulations. For the initial nominally balanced model, the modification in placement parameters was expected to compensate mainly for the location of the PCL since the collaterals were of equal length and mirrored about the sagittal plane. This simulation recommended modifying the posterior tilt of the tibial component (which mainly affects the PCL behavior), slight modifications in the translation of the femoral and tibial components and little modification of the varus/valgus and rotational alignment of the components.

TABLE 3
Component Parameters from Placement Simulations

| | Femoral | | | | | | |
|---|---|---|---|---|---|---|---|
| | Component | Femoral Component | Femoral Component | Femoral Component | Tibial Component | Tibial | Tibial Component |
| | Varus/Valgus | Internal/External | Anterior/Posterior | Proximal/Distal | Varus/Valgus | Component | Proximal/Distal |
| | Angle | Rotation | Displacement | Displacement | Angle | Posterior Tilt | Displacement |
| Simulation | (degrees) | (degrees) | (millimeters) | (millimeters) | (degrees) | (degrees) | (millimeters) |
| Initial Guess | 0 | 0 | 0 | 0 | 0 | 0 | 25 |
| Balanced | −0.2 | 0.1 | −0.5 | 0.6 | 0.2 | 2.7 | 24.3 |
| initial model | | | | | | | |
| MCL 5 mm | 0.7 | −0.4 | −1.0 | 0.2 | 4.2 | −2.6 | 22.2 |
| shorter | | | | | | | |
| PCL 5 mm | −1.0 | 1.0 | −0.7. | 1.4 | 1.3 | 3.1 | 22.6 |
| shorter | | | | | | | |
| MCL and | 0.5 | −0.7 | −1.7 | 0.6 | 4.2 | 2.6 | 21.7 |
| PCL 5 mm | | | | | | | |
| shorter | | | | | | | |
| MCL 5 mm | −1.4 | 1.5 | −0.1 | 1.5 | −3.1 | 1.9 | 25.5 |
| longer | | | | | | | |

When the MCL was shortened in the first imbalanced simulation (representing a varus imbalance), the placement of the components shifted to accommodate the imbalance. A large varus/valgus modification is needed to reduce the tension in the MCL which would occur if the components were placed for optimal alignment and this is seen primarily in the tibial component placement. The tibial component was also translated in the distal direction, thereby reducing the distance between the origin and insertion of the ligaments when the components are in contact. The tibial component was tilted anteriorly (as indicated by the negative value), which though somewhat unexpected and perhaps not clinically realistic due to the simplified anatomy, was in fact appropriate for the model, given the goal of improving ligament isometry.

The remaining three imbalance simulations resulted in appropriate modifications to the component placements. Of particular interest were the results of the elongation of the MCL by 5 mm. Although the varus/valgus angles of the components were, as expected, significantly modified, the modifications were not simply the negative of those seen in the MCL-shortened case. Here more of the imbalance was accounted for by the femoral component and an increase in the internal/external rotation of the femoral component. Without an explicit optimization process, it would be difficult to predict the appropriate changes in component placement using rules of thumb alone.

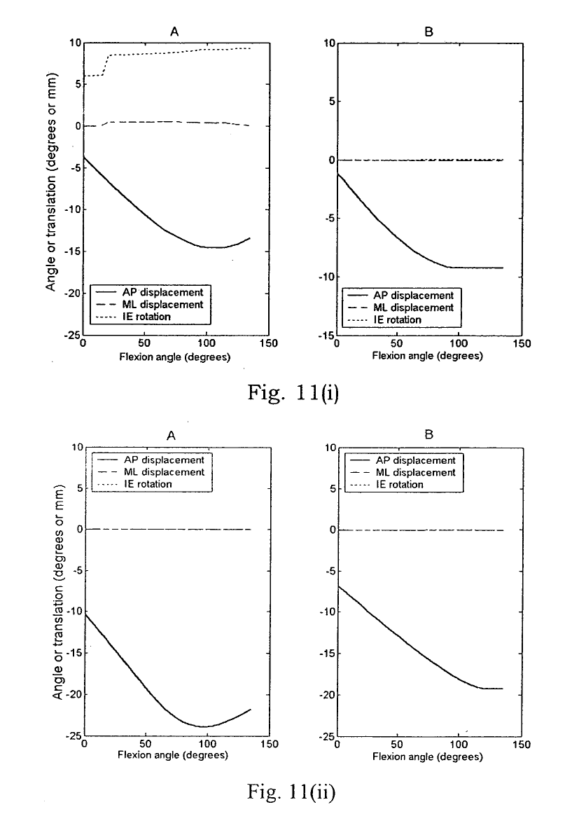

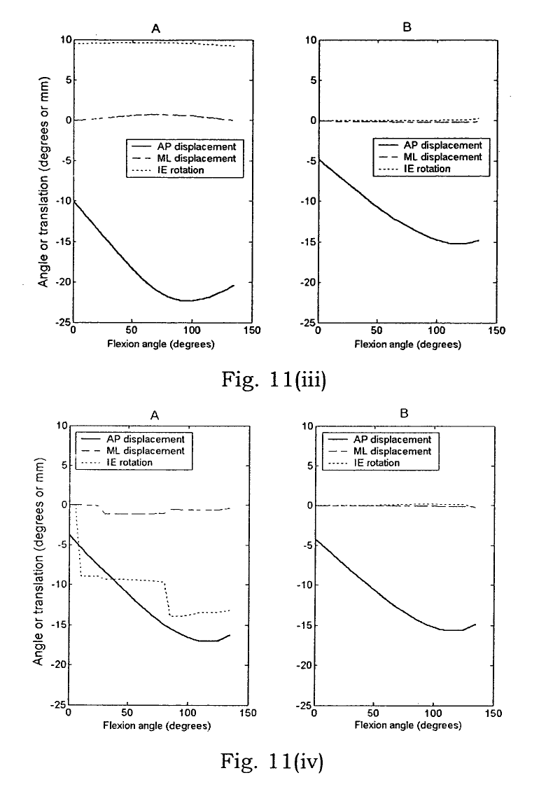

FIGS. 11(i) to 11(iv) show the passive kinematics of the femoral component with respect to the tibial component before and after the placement algorithm for all four imbalanced simulations. The “A” panels illustrate standard placements and the “B” panels illustrate the placements determined by optimization. The various situations are as follows: i. MCL shortened by 5 mm (varus deformity); ii. PCL shortened by 5 mm (flexion contracture); iii. MCL and PCL shortened by 5 mm (complex contracture); and iv. MCL lengthened by 5 mm (valgus instability). Each plot shows the anterior/posterior translation, medial/lateral translation and internal/external rotations as a function of flexion angle. For all four simulations, the final kinematics are very similar and reasonably approximate the kinematics of true knees. For example, the femoral component exhibits rollback on the tibial component. However, because of various simplifications in the models of the components and ligaments (e.g., the symmetric locations of the collateral ligaments), other normal features such as the screw-home effect are absent. The predicted kinematics prior to running the placement algorithm exhibit occasional discontinuities due to the inability of the passive kinematic model to account for slack in knee ligaments. When the knee is unstable, there is no unique or well-defined solution for its orientation. We see that after the placement algorithm has been implemented, the discontinuities are virtually eliminated and the kinematics are consistent with a stable configuration.

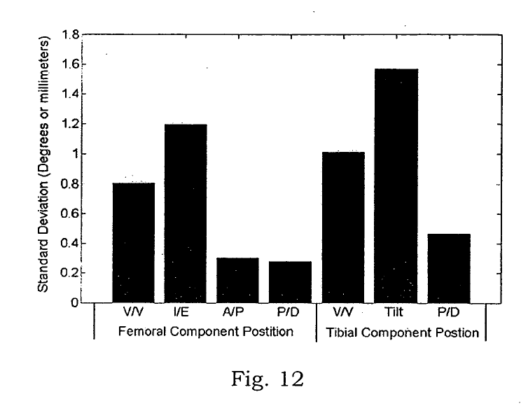

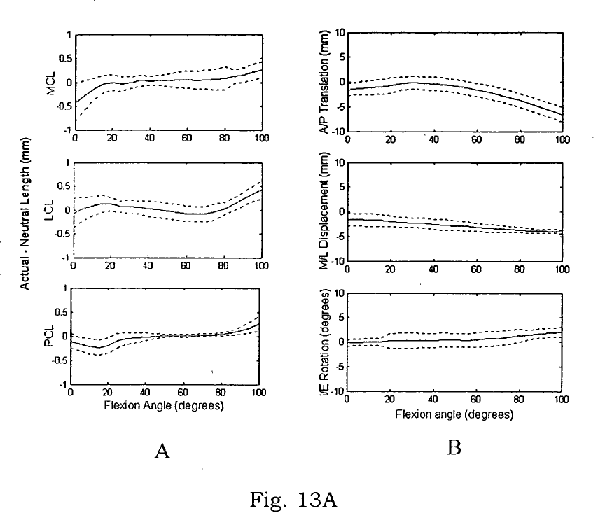

Sensitivity Analysis. The variances in placement parameters determined from the thirty trials on the porcine specimen are shown in FIG. 12. The average standard deviation for all parameters was 0.8 mm. The variance in deviation from neutral length of each ligament over the 30 placements is shown in FIG. 13A. For all three ligaments the standard deviation in ligament length was generally less than 0.4 mm for all flexion angles. FIG. 13B shows the variance in kinematics predicted by the passive kinematic model over all 30 placements. The kinematics had a standard deviation of 2 mm or less for all three of the parameters presented for all flexion angles and the trends were consistent across all trials. These results show that the placement algorithm is robust as it converges to repeatable placements and the predicted kinematics are not substantially altered by the variance of the ligament measurement technique.

The Examples described herein illustrate actual results obtained in certain specific embodiments of the invention and are not intended to represent the only results that may be obtained in all practices thereof. Moreover, the methods described herein are generally applicable to any articulating joint between first and second bones. Accordingly, the invention may also be practiced in the context of ankle, hip, elbow and shoulder surgeries, by merely applying variables appropriate for those specific joints in a corresponding manner as disclosed herein with respect to a knee joint.

The general method for determining soft tissue constraints for positioning an artificial joint includes resecting an end segment from the first bone of the articulating joint to provide space for relative movement of the two bones (in some circumstances, soft tissue resection alone may allow for this movement) and for providing an initial estimate of an attachment site for at least two ligaments attached to the first and second bones. Tension is drawn on the ligaments attached to the first and second bones and while maintaining the tension, attempts are made to move the first bone in a plurality of different directions relative to the second bone. From each attempted movement a plurality of different displacement positions of the first bone relative to the second bone are detected and represented in a defined coordinate system. For each displacement position, a plurality of new estimates of the ligament attachment sites are made by transforming the initial estimate of the attachment sites of one bone into the defined coordinate system on the other bone. A final estimate of ligament attachment position and neutral lengths for the ligaments is calculated by minimizing deviations in distance between the plurality of new estimates of ligament attachment sites of one bone and the current estimate of the ligament attachment sites in the other bone (from which the lengths are calculated).

The general method for determining placement parameters for a prosthetic component of an artificial joint between first and second bones includes defining at least one coordinate system having an origin representing a point on the prosthetic component, and providing an estimate of attachment positions and neutral ligament lengths for ligaments that remain attached to the first and second bones, such as may be obtained from the method outlined above. An initial estimate of placement parameters for the prosthetic component is provided where the placement parameter includes at least one parameter of alignment of the prosthetic component with respect to the first and/or second bone. The first bone is placed in a plurality of different flexions angles relative to the second bone and for each of the selected flexion angles. The strain energy for the attached ligaments is then calculated, a position of the prosthetic component that minimizes a total strain energy comprised of a sum of the strain energies of the ligaments is determined, an adjustment for slackness is made, if required, to determine the total ligament deviation from neutral length, the sum of ligament deviations Lᵢ for the ligaments at the selected flexion angle is determined and a position of the prosthetic component that minimizes a weighted sum of deviations of the ligaments is calculated. Total ligament deviations for all the ligaments are determined for all the flexion angles and final placement parameters for the prosthetic component are then calculated by determining placements that minimize the total ligament deviation.

From the foregoing it will be appreciated that, although specific embodiments of the invention have been described herein for purposes of illustration, various modifications may be made without deviating from the spirit and scope of the invention.

1. A method for determining soft tissue constraints for positioning an artificial knee between a tibia and femur in a subject, comprising,
providing an initial estimate of an attachment site for at least two ligaments selected from the group consisting of medial collateral, lateral collateral and posterior cruciate ligaments
distracting the tibia to draw tension on the least two of three knee ligaments and while maintaining the tension on the at least two ligaments, moving the resected tibia in a plurality of different directions relative to a femur;
detecting a plurality of displacement positions of the tibia relative to the femur when the tibia is moved in the plurality of different directions and representing the detected displacement positions in a defined coordinate system;
determining a plurality of new estimates of the ligament attachment sites by transforming the initial estimate into the defined coordinate system when the tibia is moved to the plurality displacement positions and calculating a plurality of ligament lengths from the plurality of attachment sites; and
calculating a final estimate of ligament attachment position and neutral ligament length for the at least two ligaments, the final estimate being determined by minimizing deviations between the plurality of new estimates of ligament positions and lengths.

2. The method of claim 1 further including resecting a proximal segment of the tibia of the subject prior to distracting the tibia.

3. The method of claim 1 further including resecting a distal segment of the tibia of the subject prior to distracting the tibia.

4. The method of claim 1 further including resecting a proximal segment of the tibia and a distal segment of the subject prior to distracting the tibia.

5. The method of claim 1 wherein at least one of the tibia and femur is marked with an array of markers and the act of detecting includes detecting positions of markers in the array of markers.

6. The method of claim 5 wherein the array of markers is comprised of light emitting diodes.

7. The method of claim 1 wherein the act of providing the initial estimate includes inputting the initial estimate into a computer aided surgical system.

8. The method of claim 7 wherein inputting the initial estimate position includes placing a stylus having a light emitting diode at the position to be input and detecting the light emitting diode by an optometric detection system.

9. The method of claim 8 wherein at least one of the tibia and femur is marked with an array of markers comprised of light emitting diodes and the act of detecting includes detecting positions of markers in the array of markers.

10. The method of claim 1 wherein the act of detecting the plurality of placement positions includes detecting a position of an array of markers on the limb with an electro-optical detection system and imputing the detected position into a computer aided surgical system.

11. The method of claim 1 wherein representing the detected displacement position in the defined coordinate system includes defining at least one coordinate system F_{T} and F_{F}, wherein an origin of the defined coordinate system lies on a point of space on the tibia or the femur, respectively, and wherein the estimates of ligament attachment sites and the detected displacement positions are transformed into at least one of coordinate systems F_{T} and F_{F}.

12. The method of claim 11 further including transforming the representation from the at least one coordinate system to the other of the at least one coordinate system.

13. The method of claim 12 further including defining a third, arbitrary coordinate system different from F_{T} and F_{F} and wherein transforming the representation includes transforming the representation into the arbitrary coordinate system.

14. The method of claim 1 wherein distracting the tibia in the plurality of directions includes displacing the tibia by at least two movements selected from the group consisting of anterior/posterior movement, medial/lateral movement, flexion/extension about a line connecting a pair of origins, internal/external rotation, flexion/extension about a line connecting a pair of insertions, varus/valgus rotation and straight distraction.

15. The method of claim 1 wherein distracting the tibia in the plurality of directions includes displacing the tibia by at least three movements selected from the group consisting of anterior/posterior movement, medial/lateral movement, flexion/extension about a line connecting a pair of origins, internal/external rotation, flexion/extension about a line connecting a pair of insertions, varus/valgus rotation and straight distraction.

16. The method of claim 1 wherein distracting the tibia in the plurality of directions includes displacing the tibia by at least four movements selected from the group consisting of anterior/posterior movement, medial/lateral movement, flexion/extension about a line connecting a pair of origins, internal/external rotation, flexion/extension about a line connecting a pair of insertions, varus/valgus rotation and straight distraction.

17. The method of claim 1 wherein distracting the tibia in the plurality of directions includes displacing the tibia by at least five movements selected from the group consisting of anterior/posterior movement, medial/lateral movement, flexion/extension about a line connecting a pair of origins, internal/external rotation, flexion/extension about a line connecting a pair of insertions, varus/valgus rotation and straight distraction.

18. The method of claim 1 wherein distracting the tibia in the plurality of directions includes displacing the tibia by at least six movements selected from the group consisting of anterior/posterior movement, medial/lateral movement, flexion/extension about a line connecting a pair of origins, internal/external rotation, flexion/extension about a line connecting a pair of insertions, varus/valgus rotation and straight distraction.

19. The method of claim 1 wherein distracting the tibia in the plurality of directions includes displacing the tibia by each of anterior/posterior movement, medial/lateral movement, internal/external rotation, varus/valgus rotation and straight distraction and includes at least one of flexion/extension about a line connecting a pair of origins and flexion/extension about a line connecting a pair of insertions.

20. The method of claim 1 wherein the at least two ligaments consists of the medial collateral and the lateral collateral ligaments.

21. The method of claim 1 wherein the at least two ligaments consists of the medial collateral, the lateral collateral, and the posterior cruciate ligaments.

22. The method of claim 1 further comprising,
determining placement parameters for a prosthetic components of the artificial knee, wherein the placement parameters are selected to minimize a sum of ligament deviations on the at least two of ligaments when the prosthetic components are positioned in the knee joint according to the determined placement parameters.

23. A system for accomplishing the method of claim 1 comprising,
a computer aided surgery system (CAS) configured with an electro optical or magnetic or ultrasonic (i.e., general position measurement) input device to receive an input of the initial estimate of the position of the ligament attachment site and the displacement positions of the tibia; and the CAS system being configured with instructions to determine the plurality of new estimates and to calculate the final estimate of ligament attachment site and length of the at least two ligaments.

24. A method for determining placement parameters for at least one of a femoral and tibial component of an artificial knee comprising,
defining at least one of coordinate systems F_{f} and Fₜ, where F_{f} has an origin representing a point on the femoral component and Fₜ has an origin representing a point on the tibial component,
providing an estimate of attachment positions and neutral ligament lengths for at least two ligaments selected from the group consisting of medial collateral, lateral collateral and posterior cruciate ligaments and representing the attachment positions according to at least one of coordinate systems Ff and Ft;
providing an initial estimate of placement parameters for the femoral and tibial components, where the femoral component placement parameter includes at least one parameter selected from the group consisting of femoral varus/valgus alignment, femoral internal/external alignment, femoral anterior/posterior position and femoral proximal/distal position, and the tibial component placement parameter includes at least one parameter selected form the group consisting of tibial varus/valgus alignment, tibial tilt and tibial proximal/distal position;
selecting a plurality of flexion angles of the tibia relative to the femur and for each of the selected flexion angles;
(i) calculating strain energy for the at least two ligaments,
(ii) determining a position of the tibial component relative to the femoral component that minimizes a total strain energy comprised of a sum of the strain energies on the at least two ligaments,
(iii) determining a first sum of ligament deviations Lᵢ for the selected flexion angle, the first sum of ligament deviations comprised of a sum of deviations from the neutral ligament lengths \overline{L}ᵢ for the at least two ligaments when the position of the tibial component relative to the femoral component has been determined to minimize the total strain energy;
calculating a total ligament deviation comprising a sum of the first sum of ligament deviations determined at each selected flexion angle;
calculating final placement parameters for the at least one parameter by determining placement parameters that minimize the total ligament deviation.

25. The method of claim 24 wherein the act of calculating strain energy includes determining if at least one ligament is in a slack condition (Lᵢ≦\overline{L}ᵢ) at the selected flexion angle, and if so, selecting the position of tibial component relative to the femoral component that provides the most slack, with the proviso that the strain energy for the selected position is equal or less than the strain energy calculated for the position in the absence of slack.

26. The method of claim 24 wherein estimating initial placement parameters includes estimating parameters for each parameter in the group consisting of femoral varus/valgus alignment, femoral internal/external alignment, femoral anterior/posterior position, femoral proximal/distal position, tibial varus/valgus alignment, tibial tilt and tibial proximal/distal positions; and
wherein calculating final placement parameters includes calculating the final placement parameters for each parameter in the group of parameters.

27. The method of claim 24 further including defining a first coordinate system F_{F} having an origin representing a point on the femur and defining a second coordinate system F_{T} having an origin representing a point on the tibia, and wherein positions of the ligament attachment sites are transformed from a representation in at least one of F_{F} and F_{T} to a representation in at least one of F_{f} and Fₜ.

28. The method of claim 24 further including defining a first coordinate system F_{F} having an origin representing a point on the femur and defining a second coordinate system F_{T} having an origin representing a point on the tibia, and wherein positions of the ligament attachment sites are transformed from a representation in at least one of F_{F} and F_{T} to a representation in a third, arbitrary coordinate system.

29. The method of claim 24 wherein calculating strain energy includes representing the at least two ligaments as linear springs.

30. The method of claim 24 wherein the estimate of ligament attachment sites and neutral ligament length is accomplished by the method of claim 1.

31. A system for accomplishing the method of claim 24 comprising,
a computer aided surgery system (CAS) configured with an input device to receive an input of the positions of the tibia, the femur, the component placement parameters, the at least one of attachment site and ligament lengths for the at least two ligaments, and configured with instructions to calculate the final estimate of component parameters.

32. The system of claim 31 configured with an electro optical input device to receive an input of an initial estimate of the position of the ligament attachment site and the displacement positions of the tibia; and being configured with instructions to determine a plurality of new estimates and to calculate a final estimate of ligament attachment site and length of the at least two ligaments according to the method of claim 1.

33. A method for determining soft tissue constraints for positioning an artificial joint between first and second bones in a subject, comprising,
providing an initial estimate of an attachment site and length for at least two ligaments that attached to the first and second bones;
distracting the first bone to draw tension on at least two ligaments attached to the first and second bones;
while maintaining the tension on the at least two ligaments, moving the first bone in a plurality of different directions relative to the second bone;
detecting a plurality of displacement positions of the first bone relative to the second bone when the first bone is moved in the plurality of different directions and representing the detected displacement position in a defined coordinate system;
determining a plurality of new estimates of the ligament attachment sites by transforming the initial estimate into the defined coordinate system when the first bone is moved to the plurality displacement positions and calculating a plurality of ligament lengths from the plurality of attachment sites; and
calculating a final estimate of ligament attachment position and neutral ligament length for the at least two ligaments, the final estimate being determined by minimizing deviations between the plurality of new estimates of ligament positions and lengths.

34. The method of claim 33 further including resecting an end segment from at least one of the first bone and second bone prior to distracting the first bone.

35. A method for determining placement parameters for a prosthetic component of an artificial joint between first and second bones, comprising,
defining at least one coordinate system having an origin representing a point on the prosthetic component;
providing an estimate of attachment positions for at least two ligaments that are attached to the first and second bones;
providing an initial estimate of placement parameters for the prosthetic component, where the component placement parameter includes at least one parameter of alignment of the prosthetic component with respect to at least one of the first and second bone;
selecting a plurality of flexion angles of the first bone relative to the second bone and for each of the selected flexion angles;
(i) calculating strain energy for the at least two ligaments,
(ii) determining a position of the prosthetic component that minimizes a total strain energy comprised of a sum of the strain energies on the at least two ligaments,
(iii) determining a first sum of ligament deviations Lᵢ for the selected flexion angle, the first sum of ligament deviations comprised of a sum of deviations from the neutral ligament lengths \overline{L}ᵢ for the at least two ligaments when the position of the prosthetic has been determined to minimize the total strain energy;
calculating a total ligament deviation comprising a sum of the first sum of ligament deviations determined at each selected flexion angle; and
calculating final placement parameters for the at least one parameter by determining placement parameters that minimize the total ligament deviation.
